(19) United States
(12) Patent Application Publication
(10) Pub. No.: US 2019/0192352 A1
(43) Pub. Date: Jun. 27, 2019

(54) TRANSLUCENT BANDAGE AND METHOD FOR USING THE SAME

(71) Applicant: BIODAPTIVE ADVANCED MATERIALS, LLC, Ronkonkoma, NY (US)

(72) Inventor: Daniel Rivest, Charlotte, VT (US)

(21) Appl. No.: 16/223,493

(22) Filed: Dec. 18, 2018

Related U.S. Application Data
(60) Provisional application No. 62/609,024, filed on Dec. 21, 2017.

(51) Int. Cl. A61F 13/02

(52) U.S. Cl. CPC A61F 13/0213; A61F 13/00063; A61F 13/0226

(57) ABSTRACT

A translucent bandage includes a top film, an adhesive layer positioned below the top film, an absorbent layer below the adhesive layer and a bottom perforated layer positioned under the absorbent core for adhering the bandage to a user's skin. All of the layers are sufficiently translucent or transparent to allow a user to view a wound through the bandage. An additional layer of padding or cushioning may be included in the absorbent layer for added comfort. The translucent bandage may be applied to a user's skin and the skin under the bandage may be observed for signs of a wound or infection or signs that the bandage has become uncomfortable. If such signs are observed, the bandage may be removed to provide treatment to the skin and/or wound and a new bandage may be applied.

### CROSS-REFERENCE TO RELATED APPLICATIONS

The present application claims benefit of and priority to U.S. Provisional Patent Application Ser. No. 62/609,024 entitled TRANSLUCENT BANDAGE filed Dec. 21, 2017, the entire content of which is hereby incorporated by reference herein.

### BACKGROUND

### Field of the Disclosure

The present invention relates to a translucent bandage, preferably for use in treating or preventing pressure ulcers and a method for using the same. In particular, the present invention is directed to a translucent bandage including an absorbent core layer that is adhered to a user's skin adjacent to a wound via a perforated silicone adhesive layer, where all layers of the bandage are sufficiently translucent to allow viewing of the patient's skin and the wound through the bandage.

### Related Art

Pressure ulcers are a relatively common occurrence in patients who have limited mobility and/or are confined to a bed. In order to help such ulcers heal and to avoid infection, they should be covered with bandages and monitored closely. In some cases, bandages may be used to prevent pressure ulcers from forming in the first place and thus are often used to do so in patients that are at risk. Continual removal of the bandage to observe the ulcer or wound may further irritate the wound and increase the chances of infection since the more times a bandage is moved and replaced, the more likely infection is to occur.

In addition, pressure ulcers and other wounds often produce liquid as they heal, which must be drawn away from the ulcer or wound. Thus, a bandage used to cover a pressure ulcer (or other wound) should be absorbent to soak up the liquid and allow the ulcer or other wound to dry and heal. Most conventional bandages that are absorbent are also relatively bulky and opaque such that observing the ulcer is difficult without constant removal of the bandage, which is time consuming and increases the risk of infection.

Accordingly, it would be beneficial to provide a bandage that avoids these and other problems.

### SUMMARY

It is an object of the present disclosure to provide a translucent bandage that is absorbent, stable and sufficiently translucent to allow the user's skin, including any wound covered by the bandage, to be viewed through the bandage without removal from the user's skin. Providing an absorbent and translucent bandage also allows the bandage to be placed on the user's skin even before a pressure ulcer develops as a preventative measure while still allowing for visual access to the skin to determine whether an ulcer has developed.

A bandage in accordance with an embodiment of the present application includes a translucent top film, an adhesive layer having a top surface secured to the translucent top film, an absorbent layer having a top surface thereof secure to a bottom surface of the adhesive layer and a lower layer connected to a lower surface of the absorbent layer, the lower layer including at least one opening formed therein and extending from a bottom surface thereof to a top surface thereof and including an adhesive material on the bottom surface thereof.

In embodiments, the translucent top film is made of polyurethane.

In embodiments, the adhesive layer includes a layer of adhesive applied to the bottom surface of the translucent top film.

In embodiments, the adhesive layer includes a layer of acrylic adhesive adhered to a lower surface of the translucent top film.

In embodiments, the adhesive layer includes a substrate with a layer of adhesive provided on a top surface and bottom surface thereof.

In embodiments, the absorbent layer includes a hydrogel.

In embodiments, the absorbent layer includes a hydrocolloid.

In embodiments, the absorbent layer includes: a hydrocolloid layer in contact with a top surface of the lower layer; and a cushion layer positioned above the hydrocolloid layer.

In embodiments, the cushion layer includes silicone.

In embodiments, the cushion layer includes hydrogel.

In embodiments, the cushion layer comprises a polyvinyl alcohol foam.

In embodiments, the absorbent layer includes a hydrogel and at least one active ingredient.

In embodiments, the at least one active ingredient is an antimicrobial sub stance.

In embodiments, the at least one active ingredient provides an indication of an infection.

In embodiments, the absorbent layer includes a hydrocolloid and at least one active ingredient.

In embodiments, the at least one active ingredient is an antimicrobial sub stance.

In embodiments, the at least one active ingredient provides an indication of an infection.

In embodiments, the lower layer includes a silicone based adhesive on a bottom surface thereof.

In embodiments, the lower layer includes an acrylic adhesive on a top surface thereof.

A method of treating a wound in accordance with an embodiment of the present disclosure includes: (a) providing the bandage; (b) applying the bandage to skin of a patient and over the wound; (c) inspecting the wound through the bandage; (d) removing the bandage when the inspection identifies indications of infection or indications that the bandage has become uncomfortable; (e) treating the skin of the patient; and (f) repeating steps (a) through (e).

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates an exploded view of a translucent bandage in accordance with an exemplary embodiment of the present disclosure;

FIG. 2 illustrates a cross sectional view of the bandage of FIG. 1;

FIG. 3 illustrates an exploded view of a translucent bandage in accordance with another exemplary embodiment of the present disclosure;

FIG. 4 illustrates a cross sectional view of the bandage of FIG. 3;

FIG. 5 illustrates an exemplary embodiment of a packaging element suitable to contain the translucent bandage of FIG. 1 or 2.

FIG. 6 is an exemplary flow chart for a method of treating a wound using the bandage of FIG. 1 or 2.

### DETAILED DESCRIPTION OF THE EXEMPLARY EMBODIMENTS

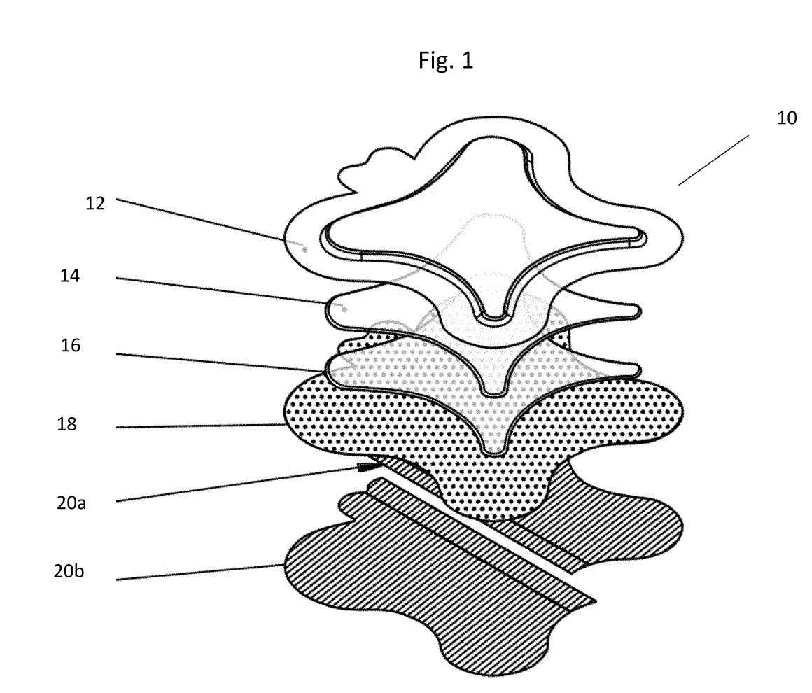

A translucent bandage 10 in accordance with an embodiment of the present disclosure is illustrated in FIG. 1. In embodiments, the bandage 10 may include a transparent or translucent top film 12. In embodiments, this top film 12 may be made of a transparent polyurethane, however, the film 12 is not limited to this material and may be made of any suitable transparent or translucent material.

In embodiments, an adhesive layer 14 is provided under the film layer 12, closer to the patient's skin when the bandage 10 is in place. In embodiments, the adhesive layer 14 keeps the film layer 12 in place, and prevents buckling of the bandage 10. In embodiments, the adhesive layer 14 may be implemented as a continuous or discontinuous coating of adhesive provided on a bottom (lower) surface of the film 12, rather than as a separate layer of material. In embodiments, the adhesive layer 14 may be made of or include acrylic adhesive, but any suitable adhesive may be used. In embodiments, as indicated in FIG. 1, the adhesive layer 14 may be a separate layer including a substrate with an adhesive material provided on a top (upper) and bottom (lower) surface thereof. In embodiments, the adhesive may be applied in a pattern using a roller and template. In embodiments, different patterns may be used.

In embodiments, such as illustrated in FIG. 1, the adhesive layer 14 may be used to connect the film layer 12 to the absorbent core, or layer 16. An advantage of using this configuration is that the adhesive layer 14 prevents bunching or folding of the bandage 10 during application and use.

In embodiments, the absorbent layer 16 may be provided under the adhesive layer 14 and adhered thereto. In embodiments, the absorbent layer 16 may be made of a hydrogel or a hydrocolloid and absorbs moisture from the ulcer or wound to which the bandage 10 is applied. While a hydrocolloid or a hydrogel are preferred materials to be included on the layer 16, other suitably absorbent materials may be used, provided that they are sufficiently translucent or transparent to allow the wound or ulcer to be viewed through them. In embodiments, silicone in combination with superabsorbent polymers or fibers may be used to form the absorbent layer 16, or a portion thereof. In embodiments, the absorbent layer may include polyvinyl alcohol foam. In embodiments, the absorbent layer 16 may be made of or include other materials provided that it is sufficiently absorbent to absorb substantial fluid from the ulcer or wound without requiring replacement, since frequent removal of the bandage 10 increases the risk of infection. In embodiments, the absorbent layer 16 may be sufficiently absorbent to absorb fluid from a wound continuously. Since wounds excrete fluid at different rates depending on the condition of the wound, bandages may need to be changed more frequently depending on the rate of healing of healing of the wound. In embodiments, a caregiver may monitor the status of the bandage 10 and will change it after it has absorbed sufficient fluid such that it swells to the point that it is uncomfortable to the patient. In embodiments, the patient themselves may monitor the status of the bandage 10 and determine when it becomes uncomfortable. In embodiments, the bandage 10 may be removed where observation indicates that a wound is not healing or where a wound is developing for treatment and a new bandage may be applied. In embodiments, the absorbent layer 16 may include one or more active ingredients. In embodiments, the active ingredient may be a antimicrobial substance. In embodiments, the active ingredient may be an additive that provides an indication of infection in the wound. In embodiment, other active ingredients may be added to provide other features.

In embodiments, a perforated adhesive lower layer 18 is provided under the absorbent layer 16 between the absorbent material and the user's skin. In embodiments, the adhesive lower layer 18 may be implemented using a clear polyurethane film with a silicone based adhesive provided thereon to secure the bandage 10 to the user's skin. In embodiments, an acrylic based adhesive may be provided on the upper surface of the adhesive layer 18 to secure it to the absorbent layer 16 while a silicone based adhesive is provided on a bottom surface and contacts the user's skin. In embodiments, the silicone adhesive on the lower surface of the adhesive lower layer 18 adheres to the user's skin around the wound while avoiding a strong bond with the wound. In embodiments, other materials may be used to make the layer 18, provided that the adhesive that faces the user's skin is silicone based. In embodiments, the perforations 18a in the adhesive lower layer 18 allow fluid to pass from the user's skin, through the lower layer 18 and into the absorbent layer 16 where it is absorbed. In embodiments, the silicone adhesive lower layer 18 provides a stable and firm connection to the user's skin to keep the bandage 10 in place while being relatively comfortable. In addition, the silicone based adhesive allows for relatively easy removal, without damaging the wound or ulcer.

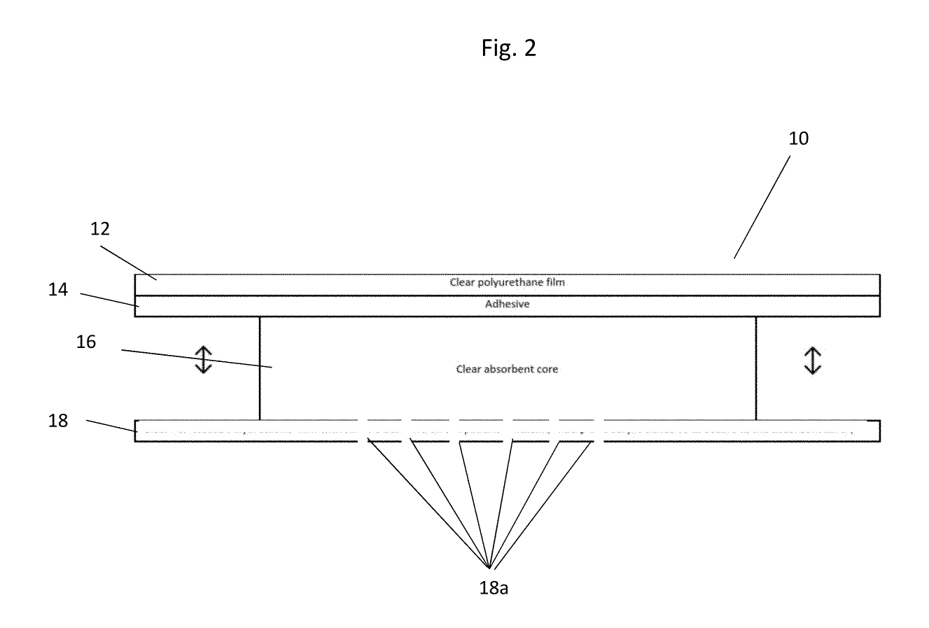

In embodiments, as can be seen in FIG. 2, for example, the bandage 10 preferably has a layered, sandwich-like structure in which the film 12, adhesive layer 14 and silicone adhesive lower layer 18 extend beyond the periphery of the absorbent layer 16 around a periphery of the bandage 10. In embodiments, all of the layers of the bandage 10 are sufficiently translucent to allow viewing of the user's skin, including a wound or ulcer under the bandage 10, without removing the bandage. In embodiments, it is not necessary that all of the layers be completely transparent, however, they must be sufficiently translucent to allow monitoring of the wound to determine how quickly it is healing and/or whether it is not healing or becoming infected. In general, wounds tend to discolor the skin and cause redness. In embodiments, the layers of the bandage 10 should be sufficiently translucent such that the reddish discoloration of a wound is visible. In embodiments, the active ingredients discuss above may tint the bandage 10, or one or more layers thereof, a certain color, blue or green, for example. In embodiments, even if the bandage 10 is tinted in color it will be sufficiently translucent to allow monitoring of the wound or skin under the bandage.

In embodiments, one or more protective liners 20a, 20b may be provided to cover the silicone adhesive lower layer 18 prior to use. In embodiments, the protective liners 20a, 20b are preferably removable from the adhesive layer 18 and are preferably made of polyethylene. In embodiments, the liners 20a, 20b may be made of a coated paper material. In embodiments, the liners 20a, 20b may be made of other materials, provided that they allow them to be removable from the adhesive layer 18 without damaging the adhesive. In embodiments, these liners 20a, 20b may be translucent, however, need not be since they are removed before applying the bandage 10 to the wound.

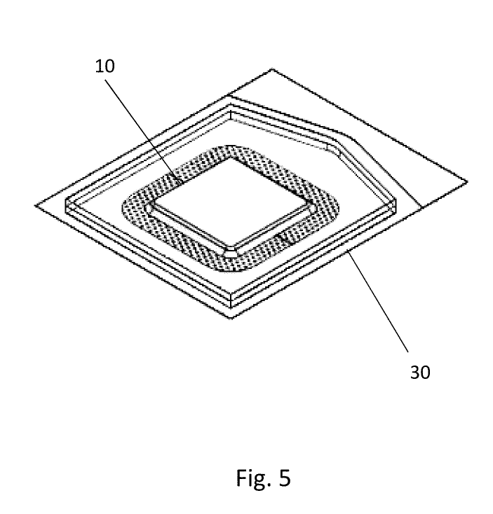

In embodiments, a packaging element 30 may be provided to store the bandage 10 before use. FIG. 5 illustrates an exemplary embodiment of the packaging element 30 in which the bandage 10 is packaged for storage prior to use. In embodiments, the packaging element 30 may be made of a combination of paper and polyethylene, or paper and paper and forms a pouch or pocket in which the bandage 10 may be stored. In embodiments, the packaging element 30 may be made of any other suitably durable material. In embodiments, the packaging element 30 may be made of a material that is suitable to allow for sterilization of the bandage 10 while in place in the packaging element. In embodiment the packaging element 30 may be made of a material that is impervious to liquids. In embodiments, the packaging element 30 may be made of a material that allows liquid to exit, but not enter, or that allows liquid to enter but not exit. In embodiments, the packaging element 30 may be transparent or include transparent portions.

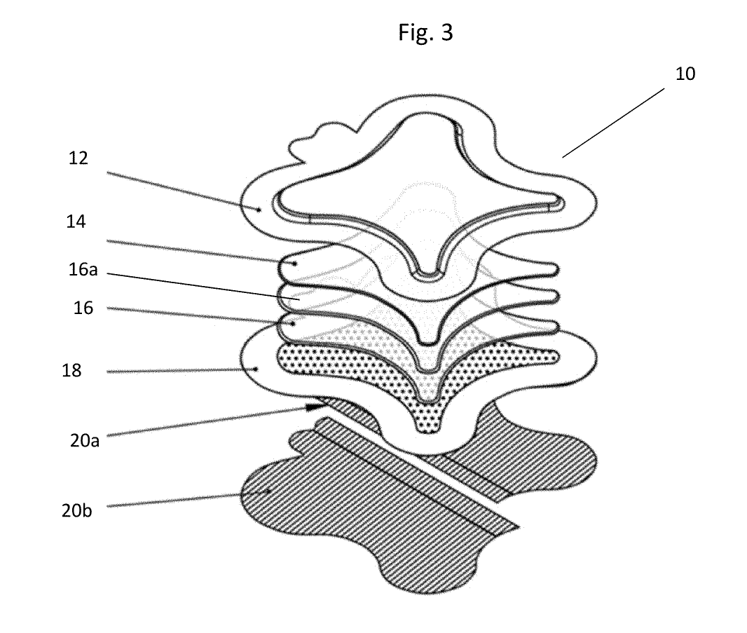

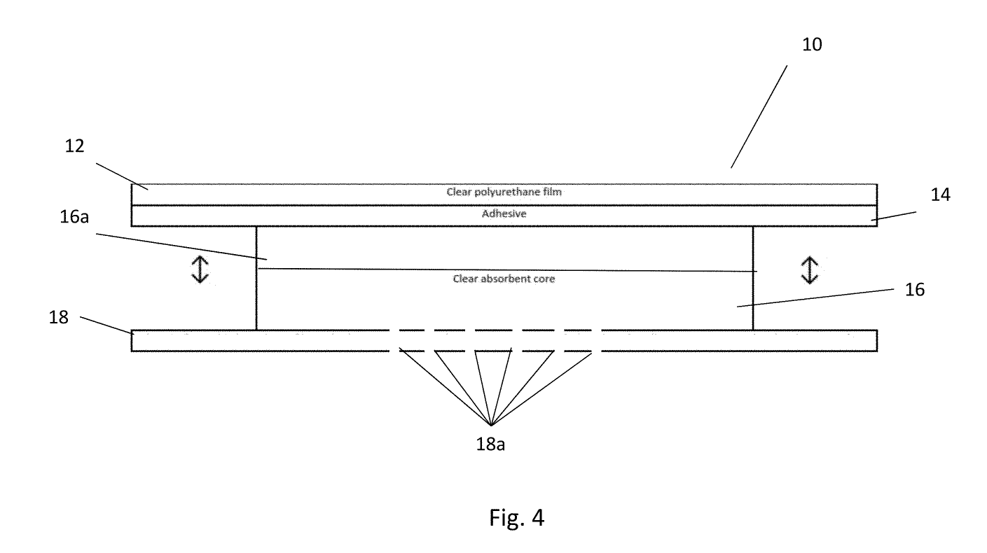

In embodiments, when the absorbent layer 16 uses a hydrocolloid material, an additional layer 16a may be provided in the absorbent layer 16 above the hydrocolloid material. In embodiments, the hydrocolloid material tends to be fairly hard, particularly as it absorbs moisture. In embodiments, the additional layer 16a may be made of a hydrogel or silicone and provides additional cushioning or padding in the bandage 10 as illustrate in the exemplary illustration of FIGS. 3 and 4, for example. In embodiments, the additional layer 16a may be made of silicone in combination with superabsorbent polymers or fibers. In embodiments, the cushion layer 16a may be made of or include polyvinyl alcohol foam. Comfort may be a key consideration for the bandage 10 since those with pressure ulcers, which are common in people with limited mobility, may not be able to adjust themselves to alleviate the discomfort of a bandage that is insufficiently padded. The additional cushion layer 16a is visible in the cross section of FIG. 4 as well as the exploded view of FIG. 3.

In embodiments, as illustrated, the bandage 10 has a butterfly like shape, however, is not limited to this shape. In embodiments, the butterfly shape, as illustrated tends to be suitable for use in the sacral area of a user's body, where pressure ulcers are common. In embodiments, the bandage 10, however, may be provided in other shapes. In embodiments, the bandage 10 may be smaller or larger than illustrated such that it is suitable for use with wounds or pressure ulcers of different sizes and on different portions of a user's body. In embodiments, the bandage 10 will be available in a variety of sizes. In embodiments, the bandage 10 will be available in sizes as small as 1.6 in.×2.5 in. and as large as 8 in.×20 in. While the cross-sectional views of FIGS. 2 and 4, for example, illustrate exemplary widths of each of the layers of the bandage 10, in embodiments, these widths may vary.

In embodiments, as noted above, the bandage 10 provides a clear absorbent core/layer 16 and utilizes a gentle silicone adhesive to allow for wound monitoring and patient comfort. In embodiments, the bandage 10 is suitable for use on full or partial thickness wounds. The bandage 10 is flexible and comfortable for the patient to wear and protects wound sites and potential wound sites while allowing for visibility so that the status of the wound may be monitored without removal of the bandage.

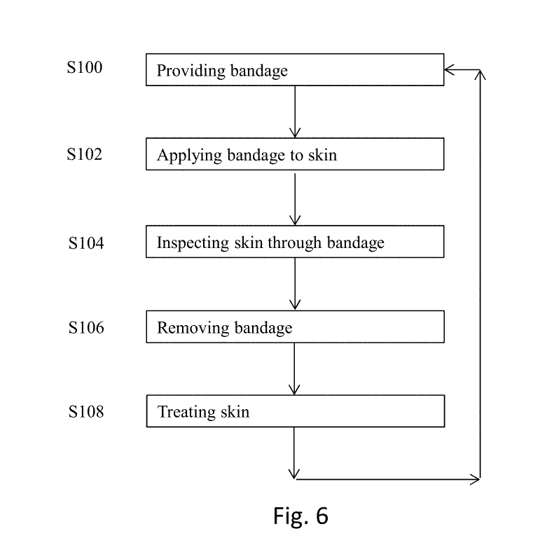

In use, in embodiments, the bandage 10 may be provided for use, for example, by removing it from the packaging element 30, in a step S100 of FIG. 6. Thereafter, the bandage 10 may be applied to a patient's skin in step S102. In embodiments, the bandage 10 may be applied over an existing wound or ulcer. In embodiments, the bandage 10 may be placed in a location where a patient is vulnerable to or otherwise likely to form an ulcer or other wound an prevent or delay formation. In embodiments, the patient or caregiver may periodically inspect the bandage 10 and the skin that is visible through it, as in step S104. In embodiments, the patient or caregiver may aperiodically inspect the bandage 10 and the skin that is visible through it. In embodiments, if the bandage 10 covers a wound or ulcer, the skin through the bandage is observed to confirm that the ulcer or wound is healing, or whether the wound or ulcer is getting worse. In embodiments, where the bandage 10 is applied prior to a wound, the skin of the patient is observed through the bandage for signs that a wound or ulcer may be developing, such as skin discoloration. In embodiments, where the caretaker or patient observes signs of wound or ulcer formation or infection, the bandage may be removed as in step S106 to provide treatment the skin as in step S108, for example. In embodiments, after treatment is provided, a new bandage 10 may be provided in the manner described above at step S100, for example. In embodiments, where the bandage 10 is absorbing fluid, the caretaker or patient may observe the thickness of the bandage to determine whether it has swollen to a point where it is uncomfortable or unstable. If so, the bandage may be removed as in step S106. Thereafter, the skin under the bandage may be treated as in step S108 and a new bandage may be provided in the manner described above at step S100, for example.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A bandage comprising:
a translucent top film;
an adhesive layer having a top surface secured to the translucent top film;
an absorbent layer having a top surface thereof secure to a bottom surface of the adhesive layer; and
a lower layer connected to a lower surface of the absorbent layer, the lower layer including at least one opening formed therein and extending from a bottom surface thereof to a top surface thereof and including an adhesive material on the bottom surface thereof.

2. The bandage of claim 1, wherein the translucent top film is made of polyurethane.

3. The bandage of claim 1, wherein the adhesive layer comprises a layer of adhesive applied to the bottom surface of the translucent top film.

4. The bandage of claim 1, wherein the adhesive layer comprises a layer of acrylic adhesive adhered to a lower surface of the translucent top film.

5. The bandage of claim 1, wherein the adhesive layer comprises a substrate with a layer of adhesive provided on a top surface and bottom surface thereof.

6. The bandage of claim 1, wherein the absorbent layer comprises a hydrogel.

7. The bandage of claim 1, wherein the absorbent layer comprises a hydrocolloid.

8. The bandage of claim 1, wherein the absorbent layer comprises:
a hydrocolloid layer in contact with a top surface of the lower layer; and
a cushion layer positioned above the hydrocolloid layer.

9. The bandage of claim 8, wherein the cushion layer comprises silicone.

10. The bandage of claim 8, wherein the cushion layer comprises hydrogel.

11. The bandage of claim 8, wherein the cushion layer comprises a polyvinyl alcohol foam.

12. The bandage of claim 1, where in the absorbent layer comprises a hydrogel and at least one active ingredient.

13. The bandage of claim 12, wherein the at least one active ingredient is an antimicrobial substance.

14. The bandage of claim 12, wherein the at least one active ingredient provides an indication of an infection.

15. The bandage of claim 1, wherein the absorbent layer comprises a hydrocolloid and at least one active ingredient.

16. The bandage of claim 15, wherein the at least one active ingredient is an antimicrobial substance.

17. The bandage of claim 15, wherein the at least one active ingredient provides an indication of an infection.

18. The bandage of claim 1, wherein the lower layer includes a silicone based adhesive on a bottom surface thereof.

19. The bandage of claim 1, wherein the lower layer includes an acrylic adhesive on a top surface thereof.

20. A method of treating a wound comprising:
(a) providing the bandage of claim 1;
(b) applying the bandage to skin of a patient and over the wound;
(c) inspecting the wound through the bandage;
(d) removing the bandage when the inspection identifies indications of infection or indications that the bandage has become uncomfortable;
(e) treating the skin of the patient; and
(f) repeating steps (a) through (e).
